(19) United States
(12) Patent Application Publication
(10) Pub. No.: US 2005/0238695 A1
(43) Pub. Date: Oct. 27, 2005

(54) Lozenge for delivery of dextromethorphan

(76) Inventors: Atma Chaudhari, Scarborough (CA); Michael Ramsay, Ajax (CA); Joseph Lee, Toronto (CA); Arthur Paul Gerald Wright, Markham (CA)

Correspondence Address: Linda A. Vag;Warner Lambert Company LLC, 201 Tabor Road, Morris Plains, NJ, 07950, US

(21) Appl. No.: 10/833,215

(22) Filed: Apr. 27, 2004

(51) Int. Cl. A61K 9/68; A61K 9/14

(52) U.S. Cl. 424/440

(57) ABSTRACT

The present invention provides an organoleptically pleasing lozenge containing an antitussive selected from the group consisting of dextromethorphan, diphenhydramine, caramiphen, carbapentane, ethylmorphine, noscapine, codeine, and mixtures thereof, complexed with an ion exchange resin wherein the particle size of the resin is 38 μm or less in diameter. Also provided is a process for producing the lozenge and methods of administering the lozenge.

### FIELD OF THE INVENTION

The present invention relates to confectionery pharmaceutical compositions containing an antitussive drug. More particularly, the invention relates to lozenges containing a dextromethorphan-resin complex. The lozenge provides a therapeutically effective dose of dextromethorphan without the bitterness or unpleasant mouthfeel associated with such lozenges.

### BACKGROUND OF THE INVENTION

Dextromethorphan (DM), is an antitussive used in many over-the-counter medications for the treatment and relief of cough symptoms associated with upper respiratory illness such as the flu or the common cold. It is commercially available in the form of its hydrobromide salt, DM-HBr (dextromethorphan hydrobromide). The salt dissolves readily in digestive juices wherein the dextromethorphan is fed into the blood stream. Biological modification and/or elimination of the medication from the body begins immediately. The usual doses therefore, for immediate release medication in the body, range from about 15 to about 30 mg administered every 4 to 6 hours.

Cough control lozenges containing a dosage of up to 15 mg dextromethorphan are available from various manufacturers. Lozenges provide convenience for consumption of a medication every 4 to 6 hours. They have the advantages of greater ease of transport and ease of administration. Dextromethorphan, however, has a bitter taste and unpleasant “mouth-feel” (i.e. the overall sensation of the product in the mouth) and is difficult to effectively mask at concentrations of greater than 2.0 mg per lozenge. In order to incorporate more than 2 mg of dextromethorphan into a palatable lozenge, an adsorbate of dextromethorphan on magnesium trisilicate (10% w/w) has been used. However, to achieve an equivalent dosage of dextromethorphan about ten times the weight of dextromethorphan adsorbate must be added. That is, the standard 3 g lozenge requires 150 mg of adsorbate to deliver 15 mg of DM-HBr equivalents per lozenge. Incorporation of this amount of adsorbate into a candy base results in a pasty, chalky lozenge texture with an unpleasant mouthfeel.

Controlled, sustained release dextromethorphan/resin complexes using ion exchange resins such as Amberlite IRP-69 (Rohm and Haas) have been developed. U.S. Pat. No. 6,001,392, for example, provides a 1:1 complex wherein no more than two times the weight of resin complex is needed to achieve an equivalent dosage of dextromethorphan. These complexes however, are used to provide controlled and sustained release from quickly consumed pharmaceutical delivery forms, in particular, liquid forms such as syrup suspensions. For these delivery forms the tastemasking of the drug need only be sufficient for this purpose.

Lozenges by their very nature are intended to slowly dissolve in the mouth over a relatively long period of time, e.g. usually about two to fifteen minutes or more, as needed. The tastebud and olfactory senses are able to detect even the slightest bitterness or unpleasant mouthfeel during this dissolution. Thus, to produce a product which overcomes both unpleasant mouthfeel and taste during such long residence time in the mouth, represents a substantial challenge.

It is desirable to provide a palatable lozenge dosage form of dextromethorphan. It is also desirable to provide such lozenges capable of delivering various amounts of dextromethorphan, and in particular, amounts from about 5 mg to about 30 mg of DM-HBr equivalents per lozenge, without the bitterness, pastiness and/or generally unpleasant taste and mouthfeel of known lozenges and delivery systems for the drug. The present invention is directed toward these and other such advantages.

### SUMMARY OF THE INVENTION

The present invention provides an organoleptically pleasing lozenge, the lozenge including
a confectionery base;
an antitussive selected from the group consisting of dextromethorphan, diphenhydramine, caramiphen, carbapentane, ethylmorphine, noscapine, codeine, and mixtures thereof,
an ion exchange resin complexed with the antitussive, wherein the particle size of the ion exchange resin is about 38 μm or less in diameter.

Also provided herein is a process for producing an organoleptically pleasing lozenge including the steps of
selecting particles of an ion exchange resin having a particle size of about 38 μm or less in diameter;
complexing the resin with an antitussive selected from the group consisting of dextromethorphan, diphenhydramine, caramiphen, carbapentane, ethylmorphine, noscapine, codeine, and mixtures thereof, as a liquid premix to form a drug-resin complex,
providing a confectionery base,
admixing the base with the drug-resin complex,
and forming lozenges containing therapeutically effective amount of the drug from the mixture.

Also provided herein are methods of administering the lozenges.

### DETAILED DESCRIPTION OF THE PREFERRED EMBODIMENTS

As used herein, dosages of DM-HBr salt complexes are referred to as “milligrams DM-HBr”. Other dosage forms which do not include the hydrobromide salt, such as ion exchange complexes (DM-resin), are referred to as “DM-HBr equivalents”. Thus, in order to be able to determine the relationship in dosage between these forms, a dosage for a dextromethorphan-resin complex will normally be referred to as “X mg DM-HBr”, with “X” being the mg of DM-HBr to which the dextromethorphan-resin complex is equivalent.

“Lozenge” is used herein to encompass slow dissolving, hard confectionery compositions which are held and dissolved in the oral cavity over a period of time, usually from about two to fifteen minutes or more, as needed. “Lozenge” therefore includes high boiled, candy confections and cold processed, sheeted candy confections (traditional lozenges).

The term “confectionery base” is used herein to mean a product containing a carbohydrate binder or bulking agent selected from a wide variety of materials such as monosaccharides, disaccharides (e.g., sucrose), polyols, oligosaccharides, polysaccharides (e.g., corn syrups and starch and the like), and in the case of sugarless bulking agents, isomalt, palatinose, palatinit and sugar alcohols such as sorbitol, xylitol, maltitol and mannitol, and the like. Such carbohydrates or bulking agents are well known to those skilled in the confectionery arts.

The term “confectionery composition” is used herein to mean a composition containing a confectionery base. In general, the base will comprise from about 5% to about 99% and preferably from about 20% to about 95% by weight of the confectionery composition.

The lozenges of the present invention provide about 5 to about 35 mg dextromethorphan per lozenge. The dextromethorphan is delivered via a dextromethorphan-resin complex wherein the resin has a particle size of less than about 38 μm (microns) in diameter. The small size of the resin complex, when used in formulations such as lozenges, confers improved mouthfeel while still preventing bitterness, as compared to lozenge formulations made with DM-HBr or with larger sized resin particles or with adsorbates such as magnesium trisilicate. The small particle size resin also provides for increased loading of the drug onto the resin with the added benefit of rapid release from the complex into the gastric juices thus providing rapid relief.

The dextromethorphan lozenges of this invention may further contain flavorants/cooling agents such as menthol, and the like, which are known for their cooling effects, i.e., throat soothing effects. The use of cooling agents provides a further benefit to the sufferer in relieving the painful sore throat symptoms which often accompany a cough and cold.

The ion exchange resins suitable for use in the dextromethorphan-resin complexes of the invention are water-insoluble and consist of a pharmacologically inert organic or inorganic matrix containing covalently bound functional groups that are ionic or capable of being ionized under the appropriate conditions of pH. The organic matrix may be synthetic or modified naturally occurring materials. Non-limiting examples of synthetic organic matrix materials include polymers or copolymers of acrylic acid, methacrylic acid, sulfonated styrene, or sulfonated divinylbenzene. Non-limiting examples of modified naturally occurring materials include modified cellulose and dextrans. The inorganic matrix may include, for example, silica gel modified by the addition of ionic groups. The covalently bound ionic groups may be strongly acidic (e.g., sulfonic acid), weakly acidic (e.g., carboxylic acid), strongly basic (e.g., quaternary ammonium), weakly basic (e.g., primary amine), or a combination of acidic and basic groups.

Any commercially available resin which may be crushed or otherwise treated to obtain a particle size of about 38 μm or less in diameter may be used. Suitable resins that may be treated in this way include Amberlite IRP-69 (available from Rohm and Haas, Philadelphia, Pa.) and Dow XYS-40010.00 (available from The Dow Chemical Company, Midland, Mich.). Each of these are sulfonated polymers composed of polystyrene cross-linked with 8% of divinylbenzene, with an ion exchange capacity of about 4.5 to 5.5 meq/g of dry resin (H⁺form). Their essential difference is in physical form. Amberlite IRP-69 consists of irregularly-shaped particles with a size range of less than 1 μm to 149 μm, produced by milling the parent, large-sized spheres of Amberlite IRP-120. The Dow XYS-40010.00 product consists of spherical particles with a size range of 45 μm to 150 μm. Another useful exchange resin, Dow XYS-40013.00, is a polymer composed of polystyrene cross-linked with 8% of divinylbenzene and functionalized with a quaternary ammonium group. Its exchange capacity is normally within the range of approximately 3 to 4 meq/g of dry resin.

Preferably, Amberlite IRP-69, a polystyrene resin, wherein the particle size has been reduced to about 38 μm or less, is used as it aids in achieving uniform dispersion, rapid release, minimal pastiness, and results in lozenges having superior taste and mouthfeel. The proper size of resin may be obtained by passing the crushed or otherwise treated resin through a 400 mesh sieve or by use of a particle classification system. The latter is often preferred for irregular shaped particles such as Amberlite IRP-69. The size of the resin can range from as low as less than 1 μm to about 38 μm.

It is preferred to size the resin prior to complexation with the dextromethorphan although the sizing may be done subsequent to the complexation step.

Complexing of the drug onto the ion exchange resin particles to form the drug-resin complex is a well known technique as shown in U.S. Pat. Nos. 2,990,332 and 4,221,778, which references are herein incorporated by reference. In general, the drug is mixed with an aqueous suspension of the resin, and the complex is then washed and dried. To achieve higher drug loadings, that is up to 65%, it is found that a multistep loading process is more efficient. That is, the drug may be divided into two or more portions, subsequent portions being mixed with an aqueous suspension of the resin-drug complex formed in the prior loading.

To achieve the desired load level an amount is used which takes into account the ion exchange process and loss of sodium bromide.

The drug-resin complex formed is collected and washed with ethanol and/or water to insure removal of any unbound drug. The complexes are usually air-dried in trays at room or elevated temperature. Adsorption of drug onto the resin may be detected by measuring a change in the pH of the reaction medium, or by measuring a change in concentration of sodium bromide by a color reaction or the drug through HPLC assay. Generally, the complexed resin particles may have up to a 20% increase in particle size and will be in the range of from less than 1 μm to about 50 μm in diameter.

Alternatively, the drug resin complex may be formed in situ in the preparation of the confectionery composition. Preparation in a prior step is preferred.

The use of particle sizes less than 38 μm provides for an increased effective total surface area per unit volume allowing for increase loading without added bitterness in the lozenge due to increased drug presence. The advantage of the increased loading is also in the reduction in the amount of resin used in a lozenge to achieve adequate dosing avoiding any unpleasant mouthfeel due to the use of resin.

The amount of the drug loaded onto the ion exchange resin may be in the range of from about 45% to about 75% by weight of the drug-resin complex. Preferably, the amount of the drug loaded onto the ion exchange resin is at least 50% and in the range from about 50% to about 75% by weight of the drug-resin complex. Most preferably, the amount of the drug loaded onto the ion exchange resin is about 55% to about 70% by weight of the drug-resin complex.

The drug-resin complex expressed as the ratio of the drug to the resin therefore is about 0.8:1 to about 3:1, preferably about 1:1 to about 3:1, most preferably about 1.2:1 to 2.3:1

The lozenges of the present invention may be used to provide drug in an amount ranging from about 5 to about 35 milligrams per lozenge.

If the average drug:resin ratio is about 1:1 (50%) an adult dose of the present invention delivered in two 3 g lozenges may contain approximately 120 mg of drug-resin complex to deliver a 60 mg DM-HBr equivalent dosage each lozenge containing a 30 mg DM-HBr equivalent dose taken every 4 to 6 hours. Alternatively, it may contain approximately 60 mg of drug-resin complex to deliver a 30 mg DM-HBr equivalent dosage each lozenge containing a 15 mg DM-HBr equivalent dose taken every 4 to 6 hours.

A preferred embodiment of the present invention provides about a 1.8:1 ratio, or 65%, of dextromethorphan loaded onto the resin. A single 3g lozenge can be formulated with only 46 mg of dextromethorphan-resin complex to deliver 30 mg of DM-HBr equivalents for adults and with 23 mg of dextromethorphan-resin complex to deliver 15 mg of DM-HBr equivalents for children. This is in contrast to use of magnesium trisilicate adsorbant, in which a standard 3 g lozenge requires 150 mg of the adsorbate to deliver 15 mg DM-HBr equivalents per lozenge

Other dosage schemes are possible as will be apparent to those having skill in the art.

Although the discussion has emphasized the use of dextromethorphan, the drug-resin complexes of the present invention are also suitable for use with other antitussive drugs and may include acidic, amphoteric or most often basic antitussives. Examples of basic drugs useful in the present invention include, but are not limited to, dextromethorphan, diphenhydramine, caramiphen, carbapentane, ethylmorphine, noscapine and codeine.

Desirably, the drug-resin complexes of the present invention have only one active ingredient, preferably dextromethorphan. In another embodiment, the invention also relates to drug-resin complexes in combination with additional pharmaceutically active compounds. Examples of such additional compounds include, but are not limited to, at least one of an antihistamine, a sympathomimetic drug (nasal decongestant, bronchodilator), an analgesic, an anti-inflammatory, a cough suppressant and/or an expectorant. Compounds which are antihistamines, sympathomimetic drugs (nasal decongestant, bronchodilator), analgesic, anti-inflammatory, cough suppressants and/or expectorants are well known to those of skill in the art, and need not be discussed in detail herein.

Once prepared, the drug-resin complexes may be stored for future use or formulated with conventional pharmaceutically acceptable carriers, to prepare the slow dissolving confectionery compositions of the invention.

The slow dissolving hard confectionery compositions, or lozenges, may be prepared by conventional methods established in the confectionery art. They may be prepared in the form of various shapes, the most common being flat, circular, octagonal and biconvex forms

The lozenges are generally of two types: high-boiled and cold processed. Preferably, the lozenge compositions of the invention are hard, high-boiled candy.

Hard boiled candy compositions have a hard texture, glassy appearance, and a solids content of 97-98%. They generally contain a confectionery base composed of a mixture of up to about 70% sugar (sucrose) and other carbohydrate bulking agents and usually up to about 92% corn syrup. They may also be prepared from non-fermentable sugars such as sorbitol, mannitol, xylitol, maltitol, isomalt, erythritol, hydrogenated starch hydrolysates and the like. Further ingredients such as flavoring agents, high intensity sweetening agents, acidulants, gelling agents, diluents, colorants, binders, humectants, preservatives and so forth may also be added.

Hard boiled candy compositions may be routinely prepared by conventional methods such as those involving fire cookers, vacuum cookers, and scraped-surface cookers also referred to as high speed atmospheric cookers. Typically, boiled candy lozenges are made by first mixing at least the carbohydrate and water and/or corn syrup in a stainless steel vessel to about 140° C. The mixture is heated until most of the moisture is driven off. The mixture is allowed to cool somewhat, and the remaining ingredients may be mixed into the batch. In the practice of the present invention it is preferred to include the dextromethorphan-resin complex at this stage in the process. Flavorants are usually added last. During the cooling process, after evaporation of moisture, the mass changes form through the liquid phase to plastic and solid. Once the candy mass has been properly tempered, it may be cut into workable portions or formed into desired shapes. A variety of forming techniques may be utilized depending upon the shape and size of the final product desired. A general discussion of the composition and preparation of hard confections may be found in E. B. Jackson, Ed. “Sugar Confectionery Manufacture”, 2nd edition, Blackie Academic & Professional Press, Glasgow UK, (1990), at pages 129-169.

Traditional lozenges are cold processed, hard confectioneries made from icing (powder) sugar, which is mixed with a binder solution, sheeted, cut to shape and allowed to dry. These lozenges tend to have a rather rough, hard finish.

Because the main ingredients of these traditional lozenges is icing sugar, the grade of sugar chosen will have a radical effect upon the final product. A fine-particle size sugar must be used; the finer the particles, the better the texture produced. If any larger particles are included, the final product will have a rough mouthfeel. The binder is usually gum arabic, gelatin, gum tragacanth, or more often a blend, in solution. Further ingredients such as flavoring agents, high intensity sweetening agents, acidulants, gelling agents, diluents, colorants, binders, humectants, preservatives and so forth may also be added.

This lozenge manufacture is a cold process. The icing sugar is loaded into a mixer such as the Z-blade type. The binder solution is gradually added to the batch and thoroughly mixed After mixing, the lozenge mix should have a firm, doughy texture. Colors and other additives including the dextromethorphan-resin complex of the present invention are also added during the mixing stage. Flavors are best added at the last possible minute. As soon as the dough is mixed sufficiently, it is loaded into a depositing hopper, extruded from the hopper into a sheet which is passed through rollers until the desired thickness is obtained. The dough is then stamped in order to cut out the lozenges, which pass onto trays, and the waste ‘web’ is reprocessed.

The lozenges are spread in a single layer on trays and allowed to form a slight crust, prior to drying in an oven at approximately 35-40° C. They are dried until their moisture content is approximately 1.5%. A general discussion of the composition and preparation of traditional lozenge confections may be found in E. B. Jackson, Ed. “Sugar Confectionery Manufacture”, 2nd edition, Blackie Academic & Professional Press, Glasgow UK, (1990), at pages 237-258.

Suitable flavorings for the hard confectionery compositions of this invention include both natural and artificial flavors, including mints such as peppermint, menthol, artificial vanilla, cinnamon, various fruit flavors, both individual and mixed, essential oils (i.e. thymol, eculyptol, menthol and methyl salicylate) and the like. Mint flavors containing menthol are preferred in a slow dissolving lozenge as menthol provides a desirable soothing effect. Cooling agents such as N-ethyl-p-menthane-3-carboxamide, 3-1-menthoxy propane 1,2-diol, and the like, may also be used to provide a cooling sensation.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. The flavorings are generally utilized in amounts that will vary depending upon the individual flavor, and may, for example, range in amounts of about 0.01 % to about 3% by weight of the final composition weight.

In an alternate embodiment of the invention the dextromethorphan-resin complex may be added to the hard confectionery composition with the aid of a lubricant. Lubricants are materials which are generally processing aids which can be mixed with the resin complex to prevent agglomeration and provide effective and uniform distribution of the complex within the lozenge. The lubricant may be present in a range of about 5 to 20% w/w with the resin complex with a range of 8 to 15% preferred. Lubricants may be selected from fats or oils or their esters or salts, waxes, mineral salts or may be synthetic polymers. Lubricants include but are not limited to fats e.g., cocoa butter, dairy fats; vegetable oils, e.g., corn oil, palm oil, coconut oil, cottonseed oil, glycerin; metal stearates, e.g., magnesium, calcium, sodium, potassium stearate; polyethylene glycols; talc; sodium lauryl sulphate; polyoxy ethylene monostearate; natural waxes, synthetic waxes, petroleum waxes; sodium salts, e.g., sodium acetate, benzoate, and oleate.

The following examples are provided to more specifically teach and better define the compositions of the present invention. They are for illustrative purposes only. The scope of the present invention is as recited by the claims that follow.

### EXAMPLE 1

### Inventive Formulation

Resin Preparation:

IRP-69 resin was placed in a 400 mesh sieve to separate out particle sizes of about 38 μm or less. The resin particles, in an aqueous suspension, were loaded with dextromethorphan in a 0.8 to 1 w/w ratio in a three-step process. To achieve a 65% loading 1580 g resin were sequentially mixed with 945 g, 650 g and 230 g of DM-HBr at 70°-80° C. for about 10 minutes. The complex was then washed and dried. The resin was assayed as 65% dextromethorphan and was used to prepare lozenges as follows.

Lozenge Preparation:

Table 1 below lists the ingredients in preparation of a 15 mg DM-HBr equivalent lozenge of the invention. Percent in the final formula is provided.

TABLE 1

| Ingredient | Grams | Final Weight % |
|---|---|---|
| Purified Water | 90.0 | 2.75 |
| Sucralose | 0.9 | 0.09 |
| Sodium Chloride USP | 1.8 | 0.18 |
| Corn Syrup | 525 | 42.2 |
| Granulated Sugar | 525 | 52.5 |
| Mono-ammonium | 4.5 | 0.45 |
| Glyyrrhizinate 20% | | |
| FD & C Red 40 | 0.11 | 0.011 |
| FD & C Blue # 1 | 0.0036 | 0.00036 |
| DM-HBr/IRP 69 resin | 6.9 | 0.69 |
| complex (Assay 65%) | | |
| Malic Acid | 6.6 | 0.66 |
| Cherry Flavor | 3.15 | 0.315 |
| 1-Menthol | 1.1 | 0.11 |
| Total | — | 100 |

Purified water, sucralose, sodium chloride, corn syrup and granulated sugar were mixed in a stainless steel vessel and heated at 140° C. until most of the water was driven off. The batch was cooled to 110° C. and the heat was turned off. 6.9 g of the drug-resin complex, mono-ammonium glyyrrhizinate, malic acid and dyes were added and thoroughly mixed into the batch. While the batch was hot and fluid, it was transferred to a cooling bench and spread into a circle. At this point, the flavoring ingredients were added. 1-Menthol was pre-dissolved in the cherry flavor before addition. The batch thickened upon cooling and was passed through a drop roller to form 3.5 g lozenges. The lozenges were allowed to cool for 15 minutes. After cooling, the lozenges were lightly sieved to remove unwanted particles before packaging. The resulting lozenge contained 2-3% moisture.

### EXAMPLE 2

### Formulation of Comparative Lozenge

Identical ingredients were used for the preparation of the comparative lozenge as were used in Example 1 but for the preparation of the drug-resin complex. The same amount of drug-resin complex was used but the drug-resin complex was prepared according to U.S. Pat. No. 6,001,392. The resin, IRP-69, was used as manufactured, i.e., particle sizes ranged from 25 μm to 200 μm. Dextromethorphan was loaded onto the resin as described above. The resin was assayed at 65% dextromethorphan

### EXAMPLE 3

### Taste Comparison

A panel of seven experts compared the lozenge of Example 1 with the comparative Example 2 for bitterness, grittiness and overall mouthfeel. The panel determined that the lozenge prepared according to Example 1 was not bitter nor gritty and, overall, had an excellent mouthfeel. In comparison, the panel determined that the level of grittiness and overall mouthfeel was unacceptable in each of the lozenges prepared according to Comparative Example 2. Additionally, four of the seven judges identified the lozenges prepared according to Comparative Example 2 as having unacceptable bitterness as well.

This test confirms that lozenges containing dextromethorphan complexed with ion-exchange resins containing particles less than 38 μm results in medicated lozenges that are less bitter and gritty than those using dextromethorphan-resin complexes of the prior art.

### EXAMPLE 4

### Immediate Release Lozenge

Table 2 below lists the ingredients in a 15 mg DM-HBr equivalent lozenge of the invention. Corn oil was used as a lubricant. Percent in the final formula is provided.

TABLE 2

| Ingredient | Grams | Final Weight % |
|---|---|---|
| Corn syrup | 290 | 42.22 |
| Granulated sugar | 290 | 52.78 |
| DM-HBr/IRP 69 resin | 4.26 | 0.78 |
| complex (Assay | | |
| 65.7%) | | |
| Fruit Juice Red Conc. | 1.0 | 0.1 |
| Water (purified?) | 40 | 2.75 |
| Sucralose | 0.2 | 0.036 |
| Mono-ammonium | 1.1 | 0.20 |
| Glyyrrhizinate 20% | | |
| Tartaric acid | 2.0 | 0.36 |
| Cherry Flavor | 0.48 | 0.89 |
| Peppermint Flavor | 0.12 | 0.22 |
| 1-Menthol | 0.50 | 0.09 |
| Corn Oil | 0.43 | 0.08 |
| Total | — | 100 |

The drug-resin complex was prepared as in Example 1. 5.0 G of the DM-resin complex was mixed with 0.43 g corn oil.

The dextromethorphan-resin complex and water were added to a mixture of corn syrup, granulated sugar and red fruit juice, mixed well, and heated to about 140° C. with intermittent mixing. To this mixture was added sucralose, mono-ammonium glyyrrhizinate-20%, and tartaric acid. The resulting mixture was mixed and allowed to cool. A premix of cherry flavor, peppermint flavor and natural menthol was added to the second mixture while it cooled. The batch thickened due to cooling and was passed through a drop roller to prepare 3.5 g lozenges. The lozenges were allowed to cool for minutes. After cooling, the lozenges were lightly sieved to remove unwanted particles before packaging. The resulting lozenge contained about 2% to 3% moisture.

1. An organoleptically pleasing lozenge, comprising a confectionery base and a drug resin complex, said drug resin complex comprising:
(a) an antitussive drug selected from the group consisting of dextromethorphan, diphenhydramine, caramiphen, carbapentane, ethylmorphine, noscapine, codeine, and mixtures thereof; and,
(c) an ion exchange resin complexed with said drug to form said drug-resin complex, wherein the average particle size of said resin is about 38 μm or less in diameter.

2. The lozenge according to claim 1 wherein said antitussive is dextromethorphan.

3. The lozenge according to claim 1 wherein said drug-resin complex delivers said drug in an amount ranging from at least 5 to about 35 milligrams per lozenge.

4. The lozenge according to claim 1 wherein a weight ratio of said drug to said resin in said complex is from about 0.8:1 to about 3:1.

5. The lozenge according to claim 1 further comprising at least one of an antihistamine, an analgesic, an anti-inflammatory, an anti-pyretic and a sympathominetic drug.

6. The lozenge according to claim 1 further comprising a lubricant.

7. The lozenge according to claim 1 further comprising a flavorant or a cooling agent or a mixture thereof.

8. A process for producing an organoleptically pleasing lozenges comprising the steps of:
(a) selecting particles of an ion exchange resin having an average particle size of about 38 μm or less in diameter;
(b) complexing said resin with an antitussive drug selected from the group consisting of salts of dextromethorphan, diphenhydramine, caramiphen, carbapentane, ethylmorphine, noscapine, codeine and mixtures thereof, as a liquid premix to form a drug-resin complex;
(c) providing a confectionery base;
(d) admixing said base with said drug-resin complex formed in step (b) to form a mixture; and
(e) forming said lozenges from said mixture.

9. The process according to claim 8 wherein said antitussive is a dextromethorphan salt or salt mixtures.

10. The process according to claim 9 wherein the dextromethorphan salt is dextromethorphan hydrobromide.

11. The process according to claim 8 further comprising adding a lubricant to said liquid premix prior to said admixing step.

12. A method of administering an antitussive drug in an immediate release lozenge said method comprising administering to a patient a lozenge of claim 1
